(19) United States
(12) Patent Application Publication
(10) Pub. No.: US 2024/0327765 A1
(43) Pub. Date: Oct. 3, 2024

(54) SYSTEM AND METHOD FOR ENHANCED WASTEWATER TREATMENT USING PHOTOTROPHIC ORGANISMS

(71) Applicant: NEWhub Holding Company, Herndon, VA (US)

(72) Inventors: Eugenio Giraldo, Robbinsville, NJ (US); Sudhir Murthy, Herndon, VA (US); Tie Mao, London (CA); Erick Wesley Cornfoot, London (CA)

(21) Appl. No.: 18/621,168

(22) Filed: Mar. 29, 2024

Related U.S. Application Data
(60) Provisional application No. 63/455,274, filed on Mar. 29, 2023.

(51) Int. Cl. C12M 1/00; C02F 3/32; C12M 1/36

(52) U.S. Cl. CPC C12M 21/02; C02F 3/322; C12M 23/22; C12M 29/14; C12M 39/00; C12M 41/06; C12M 41/48; C02F 2203/006

(57) ABSTRACT

A system and method for enhancing the wastewater treatment capacity and treatment performance of a biological wastewater treatment or biological culturing process by integrating an illuminated biofilm system with the biological process increasing the inventory of biomass and enhancing the capacity for oxygenation and/or biomass production, as well as improving nutrient removal. Photo-selection or photo-deselection of organisms by actively controlling biofilm erosion, induction of photogranulation and illumination is also presented.

### FILED OF THE INVENTION

The present invention relates to a system and method for enhancing wastewater treatment or biological culturing. More specifically, the present invention relates to illuminated method and system for growing biofilms and densified biomass within activated sludge systems or other biological wastewater treatment and biological growth methods.

### BACKGROUND OF THE INVENTION

In wastewater treatment processes, the utilization of biofilms has emerged as a promising approach to enhance treatment efficiency and pollutant removal. Biofilms, composed of microorganisms and extracellular polymeric substances, form on surfaces submerged in wastewater and play a crucial role in biodegradation and nutrient transformation. Traditional methods often rely on suspended growth processes, but biofilm-based systems offer advantages such as increased biomass retention, improved settling characteristics, and enhanced resistance to shock loads. However, conventional biofilm systems face limitations in terms of scalability, maintenance, and control over biofilm formation, prompting the exploration of innovative solutions to address these challenges.

In recent years, technologies for granulation and densification are becoming increasingly prominent. There is now an increase in interest in developing new selection or out-selection features to improve such densification or granulation. In addition, there is interest in developing new organisms that could be grown within densified biomass that could play a role in treating wastewater or to synergize with other organisms to treat wastewater.

Previous research in biofilm-based wastewater treatment has primarily focused on optimizing reactor design, substrate materials, and operating conditions to maximize treatment efficiency. Various biofilm reactor configurations, including trickling filters, moving bed biofilm reactors (MBBRs), and membrane biofilm reactors (MBfRs), have been investigated for their effectiveness in pollutant removal and nutrient recovery. Membrane biofilm reactors are a counter diffusion approach to treatment where the substrate is in the anulus of a membrane on which a biofilm is grown. Other counter diffusion approaches are of interest.

The present invention addresses the limitations of conventional biofilm-based wastewater treatment systems by introducing illuminated methods and systems to grow biofilms as a novel treatment approach. By incorporating specialized substrata that scatter light, the invention facilitates the growth of phototrophic microorganisms within biofilms, harnessing light energy to drive pollutant removal and nutrient transformation processes. This innovative approach offers advantages such as increased treatment capacity, reduced energy consumption, and improved control over biofilm formation and activity. In light of the growing demands for efficient and sustainable wastewater treatment solutions, the development of illuminated biofilm systems represents a significant advancement in the field of environmental engineering. Such growth of phototrophic organisms on biofilms could also be: a food source for higher organisms including fish and other seafood for aquaculture, or, for manufacturing proteins using other eukaryotes.

### SUMMARY OF THE INVENTION

In light of the above shortcoming, the present invention discloses an illuminating biofilm system and method for enhancing the wastewater treatment process or for growth of higher organisms that consume these biofilms.

In an aspect of the present invention, an illuminated biofilm system for enhancing wastewater treatment processes or biological culturing is disclosed. The system includes a light source, an optical coupling subsystem, a light-transmitting and scattering substratum, and a plurality of biofilm or photo-granules. The light source has a wavelength that induces at least one phototrophic microorganism growth. The optical coupling subsystem is coupled with the light source. The plurality of biofilm or photo-granules includes at least one phototrophic microorganism growing on the surface of the light-transmitting and scattering substratum. Herein, the light source and the optical coupling subsystem are enclosed and connected directly to the light transmitting and scattering substratum using electric controls, frames, supports, electric cables, optical cables, connectors, optical splitters, and other appurtenances.

In one embodiment, the illuminated biofilm system further includes an optional optical input connection connected with one of the optical coupling subsystems and the light transmitting and scattering substratum.

In one embodiment, the optional optical input connection conveys the light to the light transmitting and scattering substratum. The light transmitting and scattering substratum system induces light by illumination through the surface of the substratum forming an illuminated substratum and the scattered light through the surface of the illuminated substratum induces the growth of at least one phototrophic microorganism on the plurality of biofilm or photo-granules growing on the illuminated substratum.

In one embodiment, the illuminated substratum is submerged in a biological wastewater treatment process tank, including an activated sludge process tank.

In one embodiment, the illuminated substratum is submerged in an auxiliary vessel. The auxiliary vessel is hydraulically connected to a biological wastewater treatment process tank. An active mixed liquor exchange is present within the biological wastewater treatment process tank and the auxiliary vessel.

In one embodiment, the depth/thickness of the plurality of biofilm or photo-granules is hydraulically controlled by inducing water or gas turbulence on the surface of the plurality of biofilm or photo-granules to induce erosion of the plurality of biofilm or photo-granules or to improve diffusion characteristics of the biofilm or photo-granules.

In one embodiment, the illuminated substratum is in the shape of a fiber, or a plurality of fibers or, in the shape of a flat sheet, or a plurality of flat sheets, woven or non-woven matt, and fabric, or a plurality of woven or non-woven mats and fabrics.

In one embodiment, the mass transfer to the surface of the plurality of biofilm or photo-granules is controlled by continuous liquid movement on the surface of the plurality of biofilm or photo-granules.

In one embodiment, light is supplied continuously, intermittently, unidirectionally, bidirectionally or multidirectionally, or in pulses to the illuminated substratum.

In the preferred embodiment, the wavelength of the light source is selected to induce the growth of algae, or the growth of phototrophic bacteria, or phototrophic archaea. This growth could be induced with or without the presence of mixed liquor. Hence, this phototrophic biofilm or photogranule could host populations for near complete treatment of wastewater as a biofilm reactor system without mixed liquor similar to a biofilm reactor or photogranules with mixed liquor.

In one embodiment, the light source is selected from dimmable, intermittent based on a set pattern, operating in a pulse mode, and controlled using sensors using machine learning, and artificial intelligence approaches.

In one embodiment, material used within mobile biofilms or media from integrated fixed film activated sludge, including, but not limited to, sponges, silicone, polyurethane or other inorganic or organic material (including cellulosic or hemicellulosic material), wherein the one or more materials aid in cleaning the illuminated substratum. The biofilms or photo-granules could also be cleaned using higher eukaryotic organisms from protozoa to worms to fish and in some cases for purposeful growth of higher organisms, such as in aquaculture.

In one embodiment, the illuminated substratum is further cleaned passively or actively, including optically, biologically, chemically, or physically.

In one embodiment, light irradiates into the tank or vessel, and the irradiated light is used for one of: a) select for microorganisms and morphologies that aggregate and thus increase transmissivity of light and b) to inhibit microorganisms using photo deselection, and to grow such photo-inhibited microorganisms sheltered away from the photo source and internally contained within dense morphologies. Herein, the light irradiation could occur from an external light source located above the water including, light strips. Herein, the selection results in the accumulation of storage products as an internal carbon source, including polyhydroxyalkanoate, glycogen, carbohydrates or proteins, for denitrification or phosphorus removal or as a food for higher organisms. Herein, the selection is used to grow only sufficient phototrophs for producing oxygen necessary for aerobic reactions including for nitrification or deammonification or biological phosphorus removal or for growth of aerobic organisms.

In one embodiment, an active turbulence is use to control the size of the aggregates and the active turbulence is created by including, a pump, a mixer, a cyclone, baffles.

In one embodiment, iron compounds are added to the system.

In one embodiment, the light is irradiated to an auxiliary vessel. The auxiliary vessel is hydraulically connected to a biological wastewater treatment process tank. An active mixed liquor exchange 8 occurs with the biological wastewater treatment process tank and the auxiliary vessel.

In one embodiment, the mixed liquor is separated from a

bulk-solids medium (using a liquid-solid separator including a classifier, a lamella, a clarifier, a filter, a membrane) to improve clarity and to promote photo radiation. A phototrophic system or method for biofilm growth or granulation/densification could be placed between two solid-liquid separators which could be any of the above separators. In one embodiment, the first liquid-solid separator will have a higher loading rate to promote densification. In one embodiment, the wasting is carried out from only one of the two liquid-solid separators (either the first or second) to effect deselection of microorganism or to promote granulation/densification.

In another aspect of the present invention, a method for enhancing biological wastewater treatment processes or biological culturing is disclosed. The method includes providing an illuminated biofilm system or photo-granules, or an external light source, submerging a light transmitting and scattering substratum in a vessel where biological wastewater treatment takes place, illuminating the light transmitting and scattering substratum with light capable of inducing the growth of phototrophic microorganisms forming an illuminated substratum, growing at least one phototrophic microorganism in a plurality of biofilm or photo-granules attached to the surface of the illuminated substratum, and optionally illuminating at least a portion of the contents of the vessel with the illuminated substratum or the external light to induce photo-granulation.

In one embodiment, the plurality of biofilm or photo-granules results in the accumulation of storage products as an internal carbon source, including polyhydroxyalkanoate, glycogen, carbohydrates or proteins, for denitrification or phosphorus removal.

In one embodiment, the plurality of biofilm or photo-granules is used to grow only sufficient phototrophs for producing oxygen necessary for aerobic reactions, including for nitrification or deammonification or biological phosphorus removal.

These elements, together with the other aspects of the present invention and various features are pointed out with particularity in the claims annexed hereto and form a part of the present invention. For a better understanding of the present invention, its operating advantages, and the specified object attained by its uses, reference should be made to the accompanying drawings and descriptive matter in which there are illustrated exemplary embodiments of the present invention.

### BRIEF DESCRIPTION OF THE DRAWINGS

The advantages and features of the present invention will become better understood with reference to the following detailed description taken in conjunction with the accompanying drawing, in which:

FIG. 1 illustrates a schematic diagram of an illuminated biofilm of the present invention;

FIG. 2 illustrates a schematic illustration of the light transmitting and scattering substratum with biofilm growing on its surface in accordance with an exemplary embodiment of the present invention;

FIG. 3 illustrates the placement of the illuminated biofilm system within an activated sludge or biological culturing process, showcasing its integration into a biological tank processing wastewater, in accordance with an exemplary embodiment of the present invention;

FIG. 3A illustrates a variant arrangement wherein the illuminated system is fully submerged in a tank, facilitating the induction of photo-granulation among the microorganisms present within, in accordance with an exemplary embodiment of the present invention;

FIG. 4 and FIG. 4A depict an alternative embodiment of the illuminated biofilm system, showcasing its integration into an auxiliary vessel with active mixed liquor exchange from an activated sludge or biological culturing system, in accordance with an exemplary embodiment of the present invention;

FIG. 5 illustrates an embodiment of the illuminated biofilm system presented as a module housing multiple illuminated biofilm units, in accordance with an exemplary embodiment of the present invention;

FIG. 6 illustrates an alternative embodiment of this invention where the illuminated biofilm unit is arranged in a series of individual frames that are mounted on a larger structural support frame;

FIG. 7 illustrates an embodiment where the aspects ratios of the structural support frames are different impacting also the individual frames or individual rod units, in accordance with an exemplary embodiment of the present invention;

FIG. 8 illustrates a light source and optical coupling unit concept with LED incorporating in one case the use of heat dissipation fins in air, in accordance with an exemplary embodiment of the present invention;

FIG. 9 illustrates a heat fin and heat pipes, connecting light sources and optical coupling units in a module above water with their ends extended into water to dissipate heat, in accordance with an exemplary embodiment of the present invention;

FIG. 10 illustrates two configurations of minimizing side-emission fiber end losses of light, in accordance with an exemplary embodiment of the present invention;

FIG. 11 illustrates the exemplary use of light strips with open space in between above a water channel, or a tank or a vessel, in accordance with an exemplary embodiment of the present invention;

FIG. 12 illustrates an external light source is used in the auxiliary vessel to induce photo-granulation, in accordance with an exemplary embodiment of the present invention; and

FIG. 13 illustrates an exemplary embodiment of the present invention illustrating the light source, the supporting frame, the illuminated fibers in bundles and means for controlling the biofilm.

Like reference numerals refer to like parts throughout the description of several views of the drawing.

### DETAILED DESCRIPTION OF THE INVENTION

Embodiments are provided so as to thoroughly and fully convey the scope of the present invention to the person skilled in the art. Numerous details are set forth, relating to specific components, and methods, to provide a complete understanding of embodiments of the present invention. It will be apparent to the person skilled in the art that the details provided in the embodiments should not be construed to limit the scope of the present invention. In some embodiments, well-known processes, well-known system structures, and well-known techniques are not described in detail. Some tanks or processing elements may be missing from a figure, but those skilled in the art would understand these needs.

The terminology used, in the present invention, is only for the purpose of explaining a particular embodiment and such terminology shall not be considered to limit the scope of the present invention. As used in the present invention, the forms “a,” “an,” and “the” may be intended to include the plural forms as well, unless the context clearly suggests otherwise. The terms “comprises,” “comprising,” “including.” and “having,” are open-ended transitional phrases and therefore specify the presence of stated features, integers, steps, operations, elements, modules, units, and/or components, but do not forbid the presence or addition of one or more other features, integers, steps, operations, elements, components, and/or groups thereof. The particular order of steps disclosed in the method and process of the present invention is not to be construed as necessarily requiring their performance as described or illustrated. It is also to be understood that additional or alternative steps may be employed.

As used herein the term “substratum” means a surface upon which a biofilm grows.

As used herein, the term “suspended growth” means ordinary biological flocs, densified biological flocs, and/or photo-granules that include, but are not limited to, microorganisms (e.g., bacteria), extracellular polymeric substances, and soluble microbial products that are suspended in water. The microorganisms are living or dead, or a combination thereof. A floc exists between a single bacterium and a fully formed photo-granules and is an open structure when compared with biofilm-coated migrating carriers and does not contain a substratum.

As used herein, the term “photoselection” or “photodeselection” is the use of light to select or out-select organisms or morphologies in order to support desired biological reactions.

As used herein the term “activated sludge” means a treatment process as described in the reference Wastewater Engineering-Treatment and Resource Recovery, 2014, Metcalf&Eddy-AECOM, Chapter 8, Fifth Edition, McGraw-Hill, including at least one biological reactor, means for aerating at least one of the bioreactors receiving the influent wastewater, means for separating suspended growth in the bioreactor from treated wastewater, means for returning the separated suspended growth to the bioreactor, and means for evacuating the treated wastewater. A multitude of alternative variations of the activated sludge process 10 exist such as but not limited to completely mixed activated sludge, plug flow activated sludge, sequencing batch reactors, SBRs, membrane bioreactors, MBR, Biological Nutrient Removal, BNR, activated sludge, Enhanced Biological Phosphorus Removal, EBPR, activated sludge, step feed activated sludge, contact stabilization activated sludge, high purity oxygen activated sludge, extended aeration activated sludge, oxidation ditch, Orbal process, intermittent cycle extended aeration system, ICEAS, Cyclic activated sludge for achieving diverse goals of treatment as described in the mentioned reference above. Someone skilled in the art will identify an activated sludge process 10 and its process variations. The activated sludge process or system is a subset of a biological culturing process or system and the use of terms activated sludge in figures and drawings can be interchanged with a biological culturing process or system.

As used herein, the word “substantially” modifying, for example, a property, a measurable quantity, a method, a position, a value, or a range, employed in describing the embodiments of the disclosure, refers to a variation that does not affect the overall recited property, quantity, method, position, value, or range thereof in a manner that negates an intended property, quantity, method, position, value, or range. Where modified by the term “substantially” the claims appended hereto include equivalents to these quantities, methods, positions, values, or ranges.

Having thus described several aspects of at least one embodiment of this invention, it is to be appreciated that various alterations, modifications, and improvements will readily occur to those skilled in the art. Such alterations, modifications, and improvements are intended to be part of this disclosure and are intended to be within the spirit and scope of the invention. Accordingly, the foregoing description and drawings are by way of example only.

In an aspect of the present invention, the present invention relates to an illuminated biofilm system 100 designed for enhancing wastewater treatment processes. The invention utilizes a specialized substratum, composed of materials such as sponges, silicone, polyurethane, and various organic or inorganic substances, to promote the growth of a plurality of biofilm. The plurality of biofilm is enriched with phototrophic microorganisms, and harnesses light energy to facilitate nutrient removal and water purification within wastewater treatment systems. Higher organisms may also be grown to improve culturing of higher organisms such as fish or other eukaryotes.

The present invention as an important embodiment applies to an activated sludge process or a biological growth process 10 in any of its variations, aerobic and anaerobic, and in more general terms to a biological wastewater treatment process that removes BOD5, and/or nutrients using biological flocs or granules (i.e., suspended growth). This invention as an embodiment also helps with the intensification of treatment which can be performed through improved densification, solid residence time (SRT) uncoupling, or by placing the photo cassette to perform a unique complementary activity in a zone (for example aerobic reactions in an anaerobic or anoxic zone or anaerobic or anoxic reactions in an aerobic zone). In this way, the size of an aerobic condition is expanded into other non-aerated zones causing intensification, or the size of anoxia is expanded causing intensification. A storage approach can be produced in zones not typically observing such storage. In the case of a growth process, the biofilm is used for growth of higher organisms.

Activated sludge processes or biofilm reactors can be modified to include biofilms or granules, as granules can be considered a special case of a biofilm. In such a case, the process is compartmentalized, and the respective bacterial forms are referred to as suspended growth and biofilms. Biofilms are a layer or layers of microorganisms (e.g., bacteria, or archaea, or algae), extracellular polymeric substances, and soluble microbial products that form on surfaces. A surface upon which a biofilm grows is known as substratum and may be any one of several materials. The physical element that is used as a substratum for biofilm growth in the present invention is illuminated, scattering light through the surface in contact with water and enabling the formation of a biofilm where phototrophic organisms can grow. A specific surface area may be expressed as a unit area divided by a material volume (L2/L3) or mass (L2/M). Biofilms are typically used for the oxidation of organic matter and/or nutrients that can diffuse into the biofilm and/or the reduction of oxidized compounds that are in contaminated water, either alone or combined with suspended growth in a bioreactor. The growth of active phototrophic organisms in the illuminated substratum 3a occurs as a result of the scatter light from the illuminated substratum 3a and the nutrients present in the water forming an illuminated biofilm. In some applications of the present invention scattered light from the illuminated substratum is transmitted to the surrounding water and growth of phototrophic organisms in aggregates occurs.

In some embodiments, the wavelength of the light is such that algae can grow in the biofilm, and oxygenic photosynthesis takes place with the production of molecular oxygen (light wavelength from 380 to 700 nm), while in other embodiments the light supplied is such that purple and green phototrophic bacteria can grow (light wavelength from 700 to 1000 nm), in which case anoxygenic photosynthesis takes place and no molecular oxygen is produced. When anoxygenic photosynthesis with reduced organic compounds occurs the phototrophic organisms will release hydrogen gas, H₂, that can further be used synergistically by other microorganisms in the biofilm. The concentration of the constituents that are present in the water in contact with the illuminated substratum 3a also have an influence in the type of phototrophic organisms that can grow in the illuminated biofilm. For example, water with high concentration of BOD5 or reduced inorganic compounds such as H₂S in conjunction with an infrared wavelength will preferentially induce the growth of phototrophic bacteria or phototrophic archaea. In yet another example if visible light is used to illuminate the biofilm algae can preferentially grow in the system conducting oxygenic photosynthesis and oxygenating the biofilm from within.

The provision and maintenance of a biofilm results in a microorganism population that intensifies wastewater treatment or biological growth or culturing processes and maximizes contaminated water treatment efficiency and consistency. Bioreactors options for this system or method include those that use only a biofilm compartment include the trickling filter (TF), rotating biological contactor (RBC), biologically active filter (BAF), moving bed biofilm reactor (MBBR), fluidized bed biofilm reactor (FBBR), granular sludge reactor (GSR), and membrane biofilm reactor (MBfR). Systems that make use of both suspended growth and biofilm compartments are commonly referred to as integrated fixed film activated sludge (IFAS) or hybrid processes such as but not limited to migrating carriers or ballasted activated sludge processes 10.

In activated sludge systems, treatment capacity is often limited by solids loading (M/T·L²) that is applied to a liquid and solid separation unit, e.g. clarifiers, membranes, filters, or flotation units. For example, a clarifier solids loading capacity is a function of the solids loading, available surface area, sludge withdrawal rate, and the solids settling characteristics (commonly measured by sludge volume index, SVI). A SVI<120 mL/g is possible as an embodiment of this invention. A stationary biofilm system can increase the inventory of biomass in an activated sludge system, increasing the treatment capacity, without increasing the loading rate to the solid liquid separation unit, effectively increasing the overall capacity of the system. Stationary biofilms usually require also an increase in the aeration capacity of the biological process in order to match the oxygenation demand associated to the increase biological activity in the process.

The illuminated algae biofilms and illuminated aggregates in this invention have the benefit of not only increasing the biomass inventory of the process but also providing oxygenation to the biofilm avoiding the need for increased aeration equipment. Additionally, phototrophic organisms store polymers during photosynthesis, both oxygenic and anoxygenic, that can be used for denitrification reactions when the illumination of the biofilm is not present. Hence, an alternating illumination and not illumination of the biofilm would enable using the stored polymers accumulated during the illuminated period to remove nitrate via denitrification reactions. Placing the illuminated biofilm at the back end of a treatment process where typically external carbon is added to the process to remove last traces of nitrogen would enable substitution of external carbon addition by internal carbon generation while at the same time increasing inventory for enhanced activity. Illuminated aggregates can move along the rest of the mixed liquor in a wastewater plant optionally having periods of illumination and darkness that are used to create microbial populations in the aggregates that enhance nutrient removal. Illumination system such as the ones presented in this invention can be located at different stages in a treatment process to control the illumination and darkness cycles of the aggregates.

The thickness of an illuminated biofilm can be actively controlled by a variety of passive and active control strategies. Passive control can be achieved for example with the use of antifouling coatings: specialized coatings that are applied to surfaces under water to prevent or reduce the accumulation of fouling organisms. These coatings typically work by releasing biocides or other chemicals that discourage the growth of fouling organisms. Examples are metal based, e.g., copper, zinc, lead, silver: silicone-based and hydrophobic coatings (make it difficult for organisms to attach): and yet another passive control is the use of enzymatic coatings (by breaking down organic material before it can accumulate on the surface, e.g., oil and grease). Light-initiated antifouling coatings which can release oxidation and biocidal agents upon exposure to light, breaking down organics and preventing biofilm formation are also available as passive biofilm control strategies. Examples are photocatalytic oxidation (PCO) coatings, photodynamic antimicrobial coatings (PDCs).

Active biofilm control can be achieved by enhancing erosion of biofilm creating turbulence of the fluid in contact with the biofilm. The intensity of the fluid contact and the resultant shear force on the surface of the biofilm can be controlled using jets of fluid directed to the biofilm substratum or fluid mixing or turbulence induced by bubbling a gas into the vessel or cassette containing the biofilm. Active biofilm control can also be achieved with mechanical cleaning: This method involves physically removing the fouling by scrubbing or pressure washing the affected surface. This can be done manually or with the use of specialized equipment such as brushes, scrapers or water jets. In addition, any floating objects such as sponges which collide with surface can remove fouling. These floating objects can be themselves biofilm support media used in wastewater treatment applications.

Ultrasonic cleaning can also be used as a way of achieving active biofilm control. This method involves using high frequency sound waves to create microscopic bubbles that can remove fouling from the surface. The bubbles created by the sound waves can loosen and dislodge the fouling without damaging the underlying surface. Yet other methods of active biofilm control are:

Chemical cleaning: This method involves using chemicals to dissolve or loosen the fouling so that it can be easily removed. Chemical cleaning agents may include acids, solvents or detergents. In addition, chemical and mechanical cleaning can be applied in combination to enhance cleaning effectiveness, such acids with scrapers;

Biological cleaning: This method involves introduction of or growth of natural organisms such as bacteria, protozoan grazers to break down and remove the fouling on surface. In one embodiment, eukaryotes are grown or cultured to feed on the biofilms produced through such illumination and to simultaneously promote cleaning;

Ultraviolet cleaning: This method involves using ultraviolet (UV)-C light (200-280 nm) which is effective in inactivating microorganisms when irradiates on target surfaces, to control fouling;

High intensity light cleaning: This method involves increasing light intensity to cause photodamage and disruption of photosynthetic processes of phototrophic organisms;

Actively controlling biofilm on the surface of the illuminated substratum 3a enables a combination of photo-selection and photo-deselection approaches part of this invention. the use of photo-selection to either select or out-select organisms or morphologies in order to support reactions in water suspensions. In one particular aspect of this invention, a device submerged or unsubmerged is used as a radiation (such as of light) source to control the growth and agglomeration of organism groups in densified structures or aggregates in a manner to promote some reactions at a morphological level such as densification for enhanced settling, or at a chemical level such as iron compounds oxidation or reduction or in general oxidation or reduction of photoactive molecules, or at a biochemical level such as oxygenic or anoxygenic photosynthesis, denitrification, de-ammonification and phosphorus removal, or inhibit other reactions such as colloidal, dispersed and flocculant morphologies, or less desirable chemical reactions. The photoselection process can also manage interbacterial transfer of plasmids or nucleic acids, or the growth or inhibition of certain organisms. In one aspect of the invention, photosource is used to grow biofilms or photo-granules to remove trace nutrients from a wastewater treatment process, including trace nitrogen or phosphorus.

The form of radiation is preferably in the form of light but could also be heat or electromagnetic or other approaches that encourage or discourage the growth of organisms where the radiation may either be part of or the entire energy source for the growth of the organisms. While much of the specification is focused on photoreactions, the use of other forms of radiations is implicit when referring to such photoreactions. The absorbed energy can be used in many forms, including creating more mass or yield of organism, or to produce organic carbon, (including but not limited to polyhydroxyalkanoates, glycogen, or unique algal/phototrophic carbons) to support other reactions such as denitrification or phosphorus removal, or to produce oxygen to support the growth of other symbiotic or non-symbiotic organisms. This carbon can either be used by different organisms to create new forms of stored carbon or directly by the phototroph for such denitrification, that are carried out by organisms in the same zone as the photoreaction or in different zones associated with the pollution mitigating bioreactions.

This approach of converting photons to chemical energy for the use in pollutant management is thus considered. In some embodiments the absorbed energy is also used to consume inorganic carbon, carbonic acid or carbon dioxide or bicarbonate, that accumulates in the liquid creating excessive concentrations that are not desirable, as is the case of pure oxygen systems or in deep tanks, here presented as an exemplary system as other exist, where excessive carbon dioxide accumulates during oxidation of organic compounds or inorganic carbon, depressing pH and inducing inhibition of nitrification or nitrogen removal processes. Yet in other applications of this invention, the absorbed energy is used to remove reduced sulfur compounds such as hydrogen sulfide, or mercaptans that are produced in anaerobic environments. The radiation can also be used to promote appropriate morphologies such as dense aggregates or densified sludge, photo-aggregates, or biofilms including through the stimulation or inhibition of organisms or clusters. The formation of such aggregates can improve transmissivity of such radiation for the growth of some organisms, while providing inhibitory conditions that force other organisms to seek shelter away from such radiation in biofilm densified agglomerates, where the outer parts of the agglomerates use the radiation as an energy source, produce byproducts, and the inner parts of the agglomerates use the byproducts while at the same time sheltering themselves from the radiation source. The use of media to assist in the absorption, refraction or reflection of such radiation is also a subject of this invention. The media if desired can be used to focus the energy, such as through the use of accessory pigments, chemiosmosis, or other such approach to improve the reactions or to ‘couple’ reactions for the organisms (such as phototrophs) to improve desired conversions, is also a subject of the invention. The use of iron compounds, ligand reactions, or catalytic reaction is particularly addressed in this invention as a way of improving the activity of the photo-biofilm or photo-aggregate or densified sludge.

For example, the reactions can extend to photohydrolysis, photofermentation, and depending on the light or radiation source, the use can have oxidative or reductive effects to react or to support or enhance reactions. Again, this photo or radiation source can be applied as an internal photosystem within the reactor, or external to a reactor, inside (submerged) the fluid (including after separating the solids), or outside the fluid. When using a radiation source external to the liquid vessel, it is of interest to reduce the formation of dispersed flocculent microorganisms that create radiation absorbance limiting radiation transmissivity in the liquid. An auxiliary vessel with means for selectively retaining said dispersed flocculent microorganisms and enhancing photo-aggregates presence is also integral part of the present invention. The use of media that help maximize radiation, such as the use of fixed or partially fixed (such as but not limited sleeves) or moving sponges or other organic or inorganic, synthetic or natural material, that simultaneously provide a habitat for organisms while cleaning the bulbs or radiation source or otherwise maximizing transmissivity are part of the invention. In one embodiment, these materials could be a way to transport such moving biofilms from the photo tank or photo zone to another tank/zone where culturing organisms (including fish or shellfish) graze and eat the biofilms (containing organisms including but not limited to algae, or diatoms) grown using the photosource for the benefit of culturing.

The device can also include static (such as baffles) or dynamic (such as mixers) approaches to move the organisms or the media or the combination of organisms and media thereof, and through such motion provide purposeful directionality to either increase or decrease in light dose and/or transmissivity, or to create shear forces in biofilms or photo-aggregates that erode, remove or detach a fraction of said biofilm or aggregate to help control their size. Here light dose is the product of light intensity (such as μW/cm²) and exposure time (seconds). The device can include the focusing of any particular form or forms of radiation including specific wavelengths or frequencies that can be continuous, intermittent, alternating or any combination of radiations. The use of such alternating and intermittent radiation or wavelengths can help support different distributions of biofilms in the suspension or in the photobiofilm. This light source can also be unidirectional, bidirectional (coming from two different or opposing directions) or multidirectional as may be convenient to support reactions or for the optimized efficiency of the photoapparatus. The use of manual or automated sensors, including photo, optical, chemical, radiation, vibration, sound, light scattering, laser diffraction, temperature, transmissivity, turbidity, etc., to detect efficiency or desired end state of the intended reaction associated with the radiation is included as a system or method of this invention. These sensors when used in an automated approach could be associated with a proportional, integral or derivative (PID) control or a dead band (upper and lower bands of a measurement) control. These sensors could also measure synergistic or antagonistic reactions, including but not limited to reactions such as for nutrient removal (oxidized nitrogen or phosphorous sensors) or dissolved oxygen production or use, or Secchi depth/distance, sludge volume index, particle size, optical density as a function of managing the radiation source. Finally, the use of solar radiation or wind energy to provide external energy for the radiation source (such as the light bulb or emitter) is part of the consideration to provide a zero-carbon emitter device.

The device can be used in any field including residential, industrial or municipal applications. The use of the device for water or wastewater treatment is considered. Any processes in treatment are part of the disclosure including but not limited to conventional activated sludge, biological nutrient removal or recovery, sequencing batch reactor, membrane bioreactor, membrane biofilm processes, or any membrane processes, filters, integrated fixed film activated sludge, moving bed biofilm reactors, mobile biofilm reactors, step-feed activated sludge process 10, modified Lutzack Ettinger process, Bardenpho process, processes using a biological selector for storage organisms (including storage within phototrophs and other non-phototrophic organisms). The grown phototrophs could also be used as a source of protein including and not limited to food production for fish and other vertebrates or invertebrates such as insects or worms.

The use of the photo or radiation device for biological culturing of higher organism in a food web is considered.

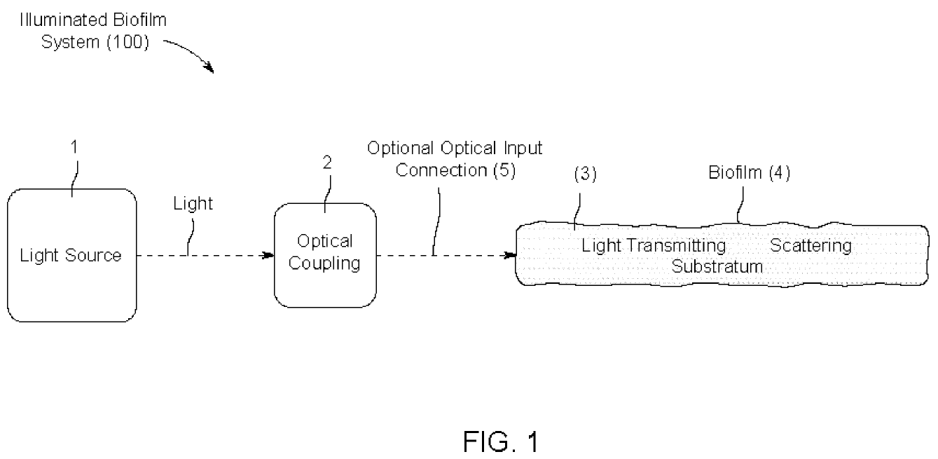

FIG. 1 is a schematic diagram of an example embodiment of an illuminated biofilm system 100 that includes a light source 1, an optical coupling subsystem 2, an optical input connection 5, a light transmitting and scattering substratum 3 and a plurality of biofilm 4 growing on the surface of the substratum 3. The light source 1 in FIG. 1 emits light that includes at least one wavelength that can be used by phototrophic microorganisms for conducting photosynthesis, either, oxygenic or anoxygenic. In some cases, the light source 1 could be sunlight whereas in other cases the light source 1 could be artificial light generated, as a matter of example, as there could be others, a lamp, a lightbulb, or a Light Emitting Diode, LED. The light from the light source 1 is optically coupled to the substratum 3 using a mechanism comprised of one or more optical elements, such as mirrors, reflectors, lenses and light pipes to collect, guide and focus the light to deliver into light substratum. Light sources 1 and their power supplies are thermally cooled by heat sinks, such as thermal fins, heat pipes exposed to cold air or water If sunlight is used, for example, the optical coupling subsystem 2 might comprise one or more mirrors or similar solar collection elements, such as but not limited to a Fresnel Lens. The optional optical input connection 5 is used to convey light from the light coupling subsystem to the light transmitting and scattering substratum 3, it is usually of a different material than the light transmitting and scattering substratum 3 with reduced light scattering.

In some embodiments the light transmitting and scattering substratum 3 is directly connected to the optical coupling subsystem 2 and no input connection is present. The light reaching the light transmitting and scattering substratum 3 is partially transmitted and partially scattered through the surface of the substratum 3 illuminating it and forming an illuminated substratum 3a. The substratum 3 consists of a core and cladding, where the core is to transmit the light through the internal of substratum, and the cladding is to scatter the light out of the substratum surface. The materials of construction of the core and cladding are different where the core has lower refractive index to allow side scattering. Optimizing the refractive index and thickness of the cladding, as well as introducing microscopic discontinuities or irregularities in the core-cladding interface can significantly increase light scattering intensity, uniformity along the surface of the substratum 3, and overall energy efficiency. While similar substratum 3 such as optical fibers for telecommunication or side-glow fibers for architectural illumination is well established using the same principle, the light transmission and scatting requirements, their wavelength dependency as well as substratum 3 form factors in this invention are distinctively different from other known applications. For example, the dimensions of the illuminated substratum 3a constrained by water treatment tanks are in meters, compared to kilometers in optical fibers for telecommunication.

The invention maximizes light scattering and uniformity and minimizes light losses through transmission. The illuminated substratum 3a induces the growth of phototrophic organisms such as algae or phototrophic bacteria or phototrophic archaea on the plurality of biofilm 4 on the surface of the substratum 3. The light source I could be dimmable, intermittent, pulsed or continuous, directional or direction modifying, to provide a required photo dose (typical units of energy/surface area). This energy could vary between 1 mJ/cm2 to 1000 mJ/cm2 depending on the light source 1 used. Other approaches are also possible to calculate the light received by organisms instead of light being dosed. Other approaches using lux or lumens are also possible. The light source 1 can be set to any pattern, controlled using sensors or using machine learning or artificial intelligence approaches. The approach of controlling this light source 1 could be using sensors that are based on any light parameter (such as but not limited to turbidity, transmissivity, light intensity, light diffraction or scattering). This parameter provides the required information on the particles in biofilm or in suspension as well as the ability of the light source 1 to influence such particles. Other sensor parameters are also possible to control the photoreaction (both stimulatory and inhibitory). One example includes the measurement of oxidized nitrogen or phosphorus parameters (such as nitrate or nitrite) to monitor the progress of reactions (such as nitrification, denitrification, deammonification or phosphorus removal) that have been assisted by the light source 1. Yet in other applications, such as, when removal of dissolved inorganic carbon species such as carbonic acid, or carbon dioxide or bicarbonate, sensors are of interest (especially for managing pH, including to increase pH), sensors to monitor said inorganic carbon species or pH or both are incorporated. Other measurements could include the use of a dissolved oxygen probe to measure the progress of oxygen production or consumption associated with photoreactions. When using the system for reduced sulfur compounds removal, such as but not limited to hydrogen sulfide or mercaptans, sensors for measuring such compounds are also considered. The use of sound waves could also be used as needed to sense and provide information on the reactions or the contained morphologies. The light source could be used for biological culturing in one embodiment. The light source could be with or without mixed liquor, with or without biofilm carriers. The pulsing of light can be used to manage growth or yield of organisms (either increase or decrease in different configurations) or to affect the culturing or to assist in the treatment.

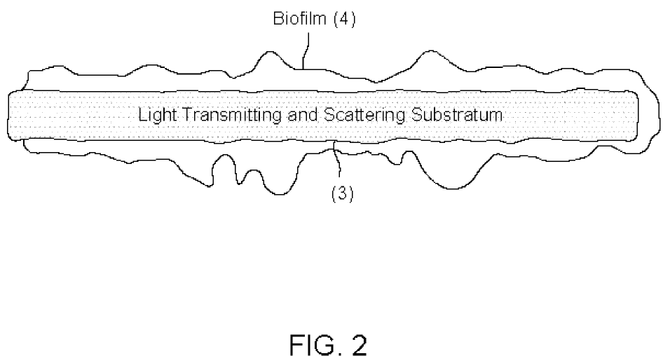

FIG. 2 illustrates one schematic illustration of the light transmitting and scattering substratum 3 with the plurality of biofilm 4 growing on its surface: the light scattered through the surface of the substratum 3 forming an illuminated substratum 3a, illuminates the biofilm and induces the growth of phototrophic microorganisms as part of the biofilm. The plurality of biofilm 4 might also contain other type of microorganisms in a symbiotic relationship with the phototrophic ones. The type of non-phototrophic organisms in the plurality of biofilm 4 will depend on the placement (including for example in different trophic or electron acceptor requiring conditions) of the illuminated biofilm within a wastewater treatment plant.

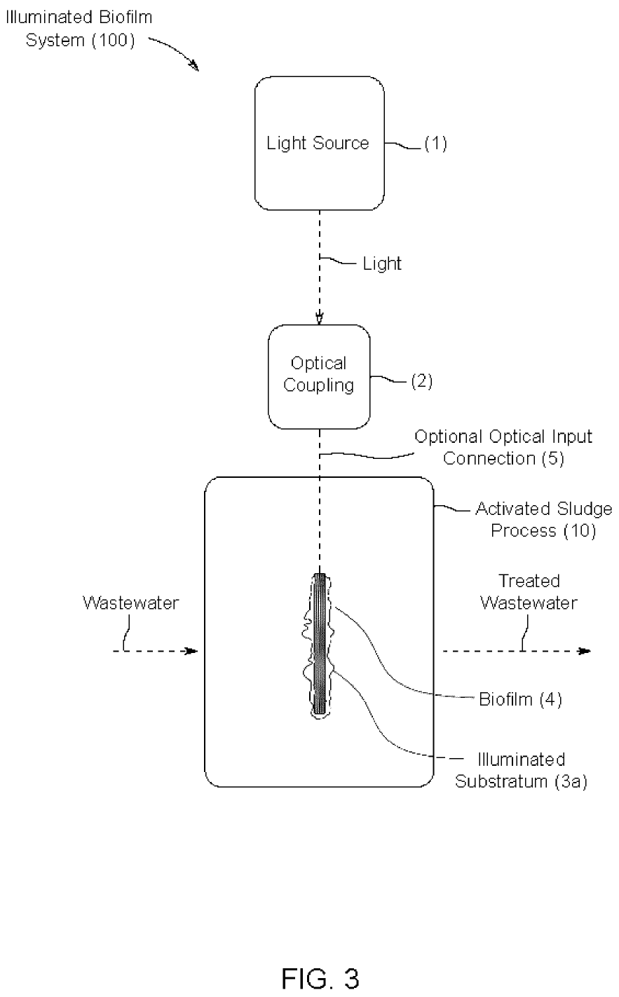

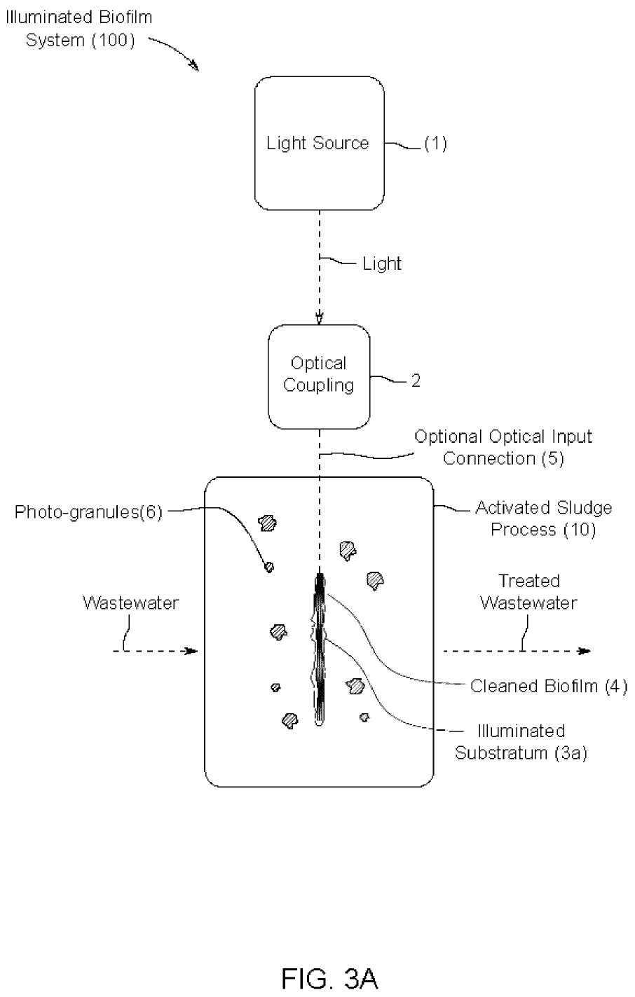

FIG. 3 and FIG. 3A are similar to FIG. 1 but illustrates the placement of the illuminated biofilm system 100 as part of an activated sludge or biological culturing process 10 as a matter of examples as the system can be placed in a biological tank processing wastewater in a system different from an activated sludge to either have a photobiofilm grow in proximity to or over the photosource (as shown in FIG. 3, or in some embodiments the illuminated system is submerged in a tank to induce photo-granulation 6 of microorganisms present in the tank as shown in FIG. 3A. It may be then required to prevent or manage the growth of biofilm 4 (to promote photogranulation or densification) on or near the photosource as illustrated in FIG. 3A by cleaned biofilm 4. The-said tank may either be directly or not directly part of the activated sludge or biological culturing process 10. In one embodiment, the liquid is separated from a bulk solid medium (using a liquid-solid separator) and this liquid, largely separated from the bulk is exposed to photo reactions (thus for external photoselection) to grow the requisite phototrophs before sending this liquid mass back to bioprocess in any preferred zone for the depolluting reaction. The liquid-solid separator, can be a classifier, a lamella, a clarifier, a filter, a membrane, as various example embodiments. In one embodiment, the photo source is placed between two liquid-solid separators. In one embodiment, the first liquid-solid separator is loaded at a higher rate than the second separator. In one embodiment, wasting for SRT uncoupling occurs from the solids obtained from only one of the two liquid-solid separators. In one embodiment, only one of the liquid-solid separators is used to recycle or return solids obtained from such separation. In one embodiment, both of the liquid-solid separators are used to recycle or return part or all of the solids obtained from such separation. Another embodiment approach is to use variable or intermittent mixers to allow for example the settlement of bulk solids (and thus effecting separation), exposure of liquid to light, and then the resumption of mixing in such manner of variable control or intermittent action.

Another approach is to use variable or intermittent mixers to allow for example the settlement of bulk solids (and thus effecting separation), exposure of liquid to light, and then the resumption of mixing in such manner of variable control or intermittent action. Yet in other embodiments the illuminated substratum 3a is submerged in at least one of the biological tanks processing wastewaters in an activated sludge or biological culturing system. Several units can be collocated in one biological tank, and one or several units can be located in different tanks through an activated sludge or biological culturing process 10. In some cases, the light used to illuminate the plurality of biofilm 4 induces the growth of algae that conduct oxygenic photosynthesis, while yet in other cases the light used to illuminate the plurality of biofilm 4 induces growth of phototrophic bacteria conducting anoxygenic photosynthesis. Photoreactions other than photosynthesis are possible as previously or subsequently described. The illuminated substratum 3a can be placed in an anaerobic biological process tank for example, while in other cases can be placed in an anoxic tank, and yet in other cases it can be placed in an aerobic tank with concentration of oxygen higher than 1 mg/L, while in other cases it can be placed in tanks with concentrations of oxygen lower than 1 mg/L, or even lower than 0.5 mg/L. In some cases, the biological tank is subject to continuous aeration while in other cases the biological tank has cyclic aeration. Yet in other cases the illuminated substratum 3a is placed in a post anoxic tank as when incorporation of nitrogen and phosphorus by photo-induced growth is desirable or formation of photo-induced polymers for nitrogen and phosphorus removal are required. It is known that radiation (for example ultraviolet) can alter a chemical (especially such as but not limited to a trace pollutant), or excite the bonds, or to promote co-reactions with other chemicals (including for example ligands and catalysts). The use of light or radiation in such a manner co-joined with a bulk bioreactor or within a bulk bioreactor is envisioned in this invention.

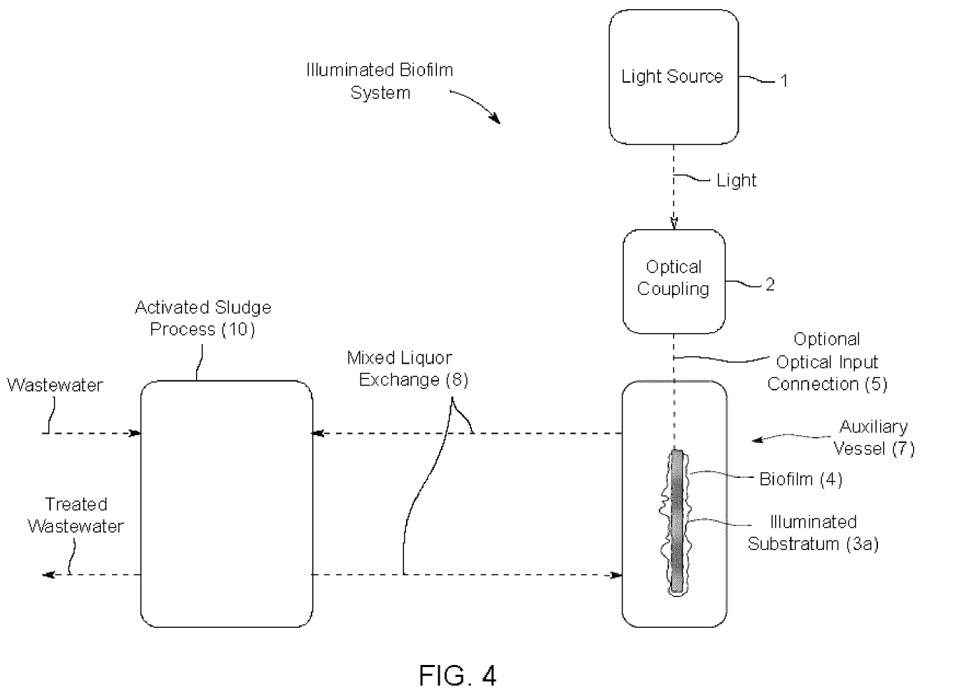

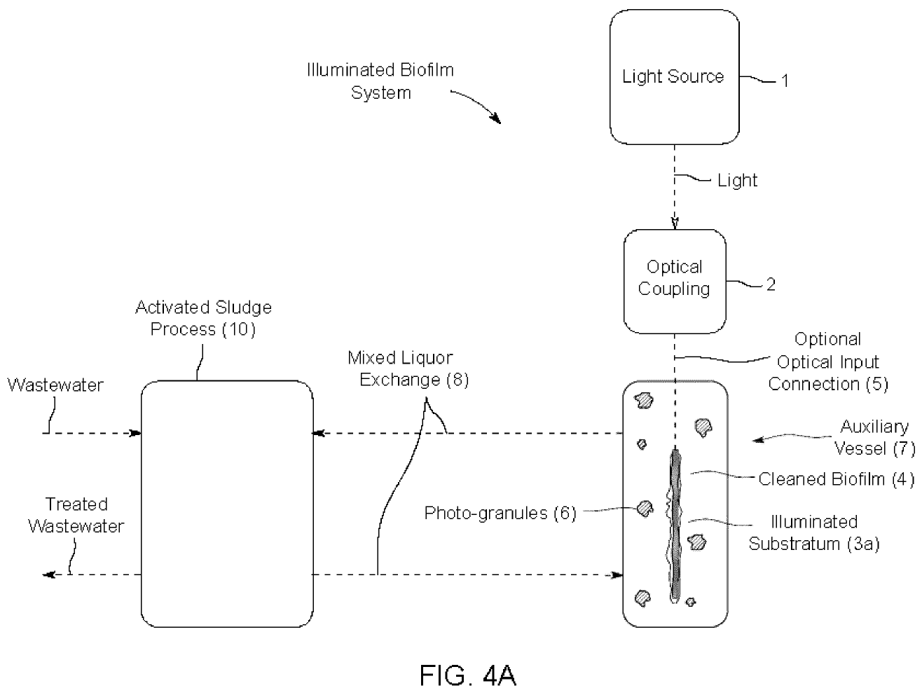

FIG. 4 and FIG. 4A illustrate yet another embodiment of the present invention where the illuminated substratum 3a part of the illuminated biofilm system 100 is submerged in an auxiliary vessel 7 that has an active exchange of mixed liquor from an activated sludge system. Someone skilled in the art will realize that the auxiliary vessel 7 with mixed liquor exchange 8 can also exchange mixed liquor from a biological tank processing wastewater in other type of wastewater treatment or culturing systems different from an activated sludge as in anaerobic processes or others. The auxiliary vessel 7 containing the illuminated substratum 3a could exchange mixed liquor of the biological tanks processing wastewater in an activated sludge system. Several units can be exchanging with a biological tank, and one or several units can be located in different tanks through an activated sludge process or biological culturing process 10. In some cases, the light used to illuminate the plurality of biofilm 4 induces the growth of algae that conduct oxygenic photosynthesis, while yet in other cases the light used to illuminate the biofilm induces growth of phototrophic bacteria conducting anoxygenic photosynthesis. The illuminated substratum 3a can be placed to exchange mixed liquor with an anaerobic biological process tank for example, while in other cases can be placed to exchange mixed liquor with an anoxic tank, and yet in other cases it can be placed exchanging mixed liquor with an aerobic tank with concentration of oxygen higher than 1 mg/L, while in other cases the concentrations of oxygen are lower than 1 mg/L, or even lower than 0.5 mg/L.

In some cases, the biological tank is subject to continuous aeration while in other cases the biological tank has cyclic aeration. Yet in other cases the auxiliary vessel 7 with the illuminated substratum 3a is placed exchanging mixed liquor with a post anoxic tank. In some embodiments the illuminated system 100 is submerged in a tank to induce photo-granulation 6 of microorganisms present in the tank (and the formation of photo-biofilms on the surface may be inhibited or prevented in order to sufficiently photo-radiate as illustrated in FIG. 4A cleaned biofilm 4, and the produced photo-granules are used to provide seed granules to the active process where exchange takes place. As part of the mixed liquor exchange 8, the said tank may either be directly or not directly part of the activated sludge process 10. In one embodiment, the liquid is separated from a bulk-solids medium (using a liquid-solid separator) to improve clarity and to promote photo radiation, and this liquid, largely separated from the bulk is exposed to photo reactions (thus for external photo-selection) to grow the requisite phototrophs before sending this liquid mass back to bioprocess in any preferred zone for the depolluting reaction. The liquid-solid separator, can be a classifier, a lamella, a clarifier, a filter, a membrane, as various example embodiments. Another approach is to use variable or intermittent mixers to allow for example the settlement of bulk solids (and thus effecting separation), exposure of liquid to light, and then the resumption of mixing in such manner of variable control or intermittent action. In one embodiment, the sludge is wasted only from the lighter fraction and the heavier fraction containing photo-granules are retained at a SRT greater than the lighter non-granular fraction. In one embodiment, the photosource is placed between two liquid-solid separators. In one embodiment, the first liquid-solid separator is loaded at a higher rate than the second separator. In one embodiment, wasting for SRT uncoupling occurs from the solids obtained from only one of the two liquid-solid separators. In one embodiment, only one of the liquid-solid separators is used to recycle or return solids obtained from such separation. In one embodiment, both of the liquid-solid separators are used to recycle or return part or all of the solids obtained from such separation. In the approach for SRT uncoupling using photo selection, the SRT of photo-granule fraction is at least 2 times the SRT of floc or non-granular fraction.

In one approach, mixing is used to bring the phototrophs in proximity to the light source 1, and jetting action or a directed flow may help simultaneously bring the phototrophs to the light source 1 while maintaining a clean surface. So rather than have quiescent conditions, a mixed approach may be facilitated as needed. Thus, mixing or lack thereof can be used to promote the plurality of biofilm 4 or photo-granules.

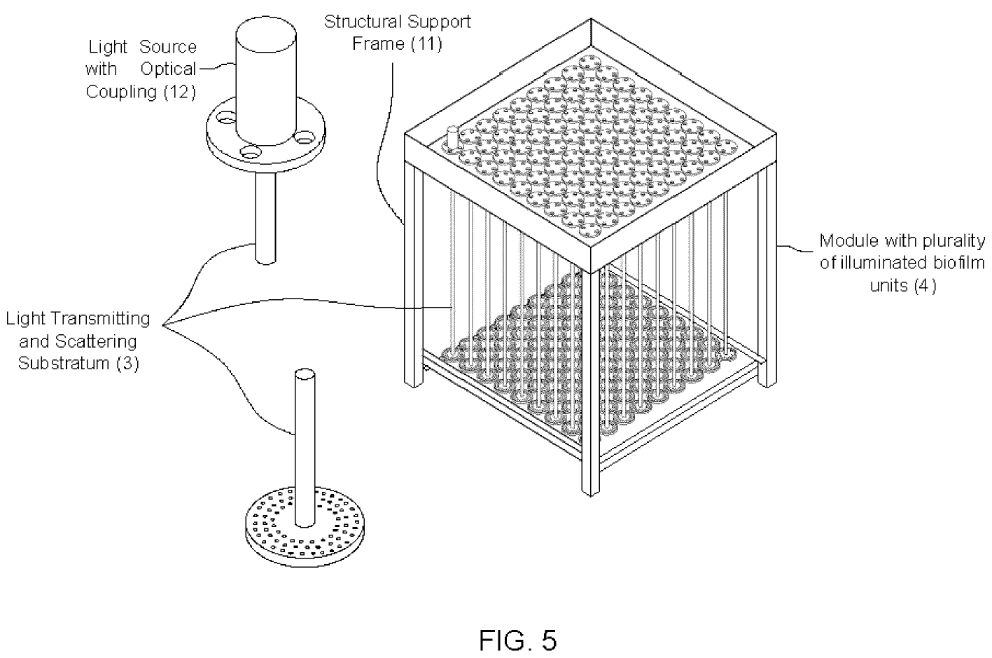

FIG. 5 illustrates an embodiment of this invention in the form of a module with a plurality of illuminated biofilm 4 units mounted where the light source and coupling unit 12 is enclosed and connected directly to the light transmitting and scattering substratum 3 in this case as fibers. The fibers can be supported around a structural support frame 11 for enhanced durability. The plurality of biofilm 4 units is mounted in the frame 11 to form a module. The wiring and electrical connections are done on the top of the frame 11 and might be connected to an electrical cabinet. The plurality of biofilm 4 unit is removable and replaceable from the top of the module and has potted ends to space the fibers. The module becomes then one unit that can be placed withing the biological process tank or the auxiliary vessel 7.

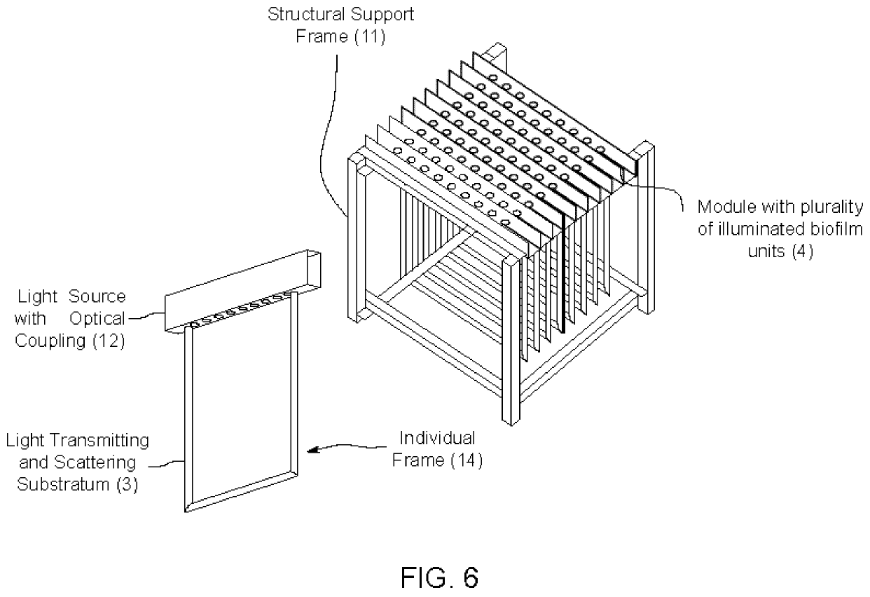

FIG. 6 further illustrates an alternative embodiment of this invention where the illuminated biofilm unit is arranged in a series of individual frames 14 that are mounted on a larger structural support frame. The individual frames 14 have the light source and optical coupling unit 12 on top connected to a plurality of light transmitting and scattering substratum 3 units, which could be fibers. The individual frames 14 are removable as a unit that could be welded at one end a with a plug at the other end. The fibers could be attached at the bottom. Each individual frame 14 might have a power cable to energize the light source 1 and local electronics and controls. In some cases, thermal dissipation devices might be necessary and incorporated in the design.

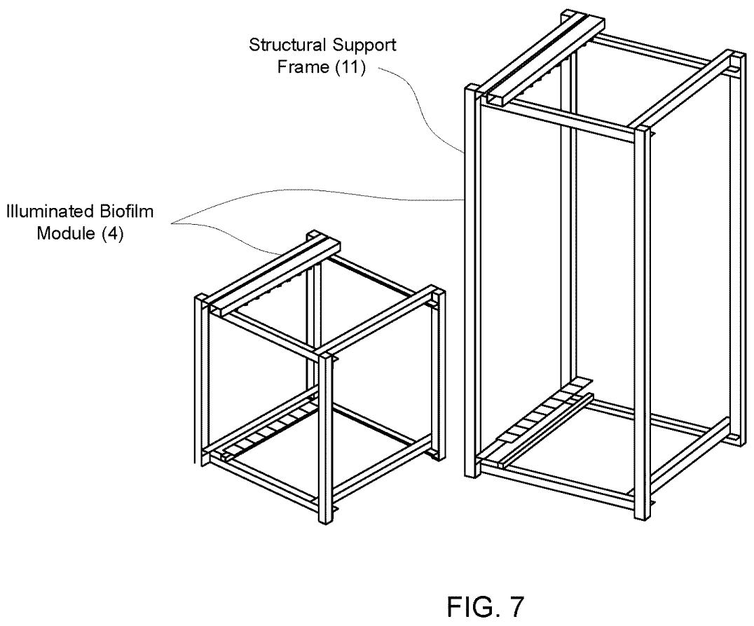

FIG. 7 further illustrates an embodiment where the aspects ratios of the structural support frames 11 are different impacting also the individual frames 14 or individual rod units. Different geometric aspect ratios might be necessary depending on management of heat and conveyance and scattering of light in the substratum 3.

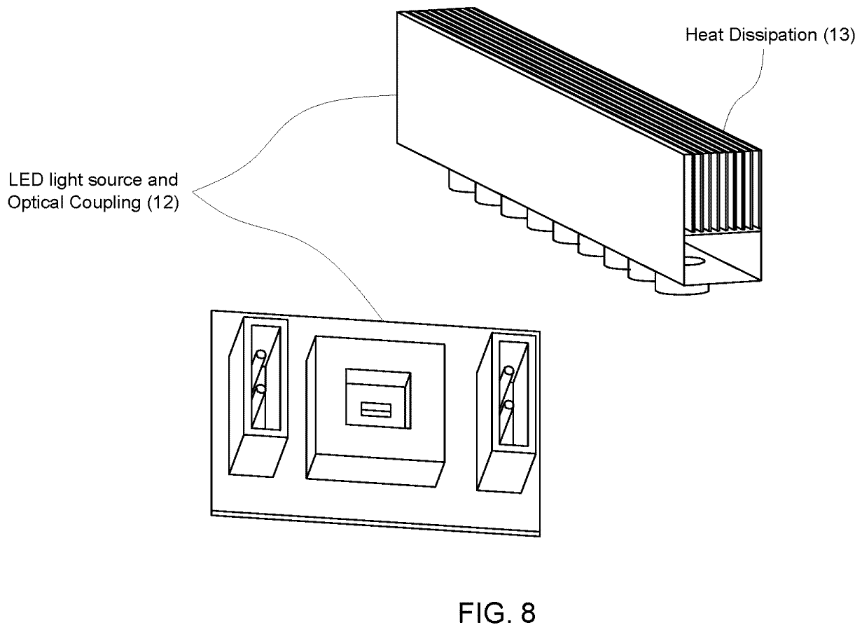

FIG. 8 Illustrates a light source and optical coupling unit 12 concept with LED incorporating in one case the use of heat dissipation 13 fins in air. An array of LEDs is located on a board, the board might incorporate local electronic controls for the unit.

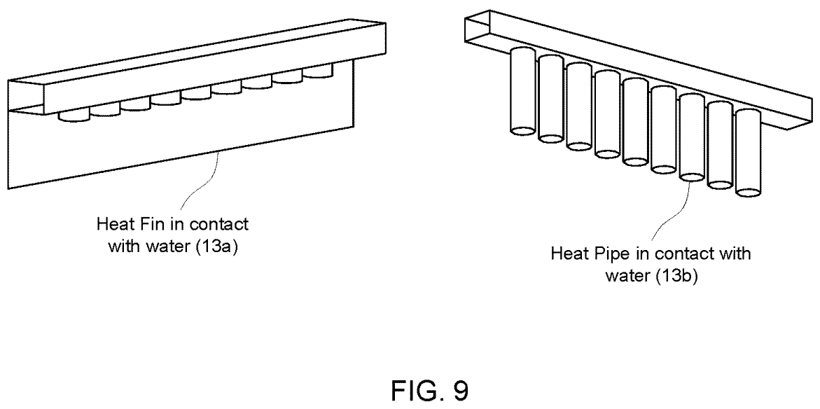

FIG. 9 Illustrates a heat fin 13a and heat pipes 13b, connecting light sources and optical coupling units 12 in a module above water with their ends extended into water to dissipate heat. Mechanical wiping or spraying of a cleaning agent on the thermally conducted surfaces may be required to maintain thermal transfer efficiency of these elements.

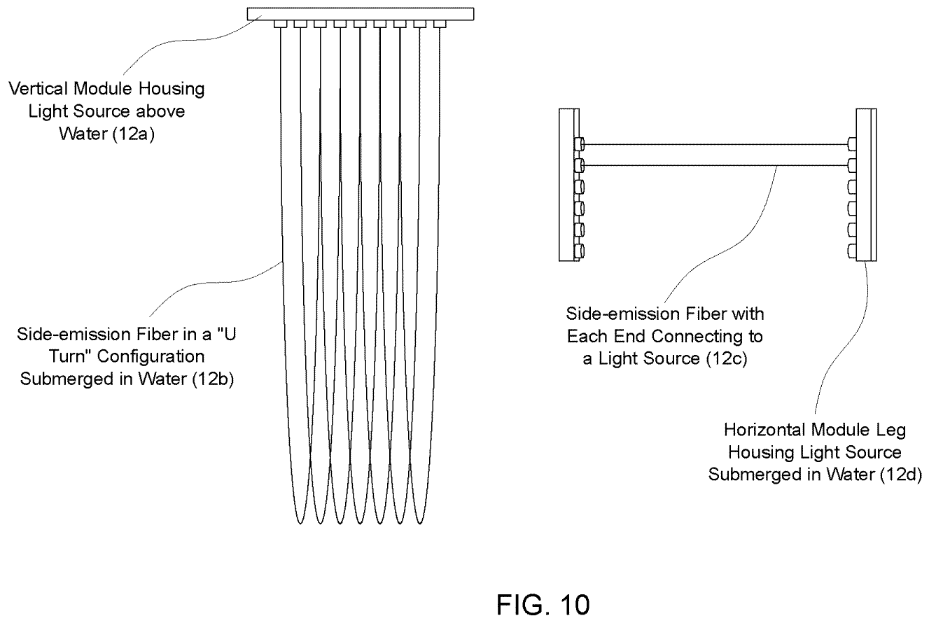

FIG. 10 Illustrates two configurations of minimizing side-emission fiber end losses of light. In one configuration, a module housing light sources and optical coupling unites connecting side-emission fibers vertically 12c. The fibers are arranged in a “U-turn” configuration 12b with one end connecting to a light source 1, and another end connecting to a different source, spaced in a way to meet fiber bending radius requirements to reduce bending light losses and mechanical stress. In another configuration, the light sources are installed in the vertical posts 12a that are submerged with the fibers running horizontally with their ends connecting to light sources. This configuration is beneficial for cooling, and the height of the module can vary without changing the length of the fiber assemblies by adding more of them.

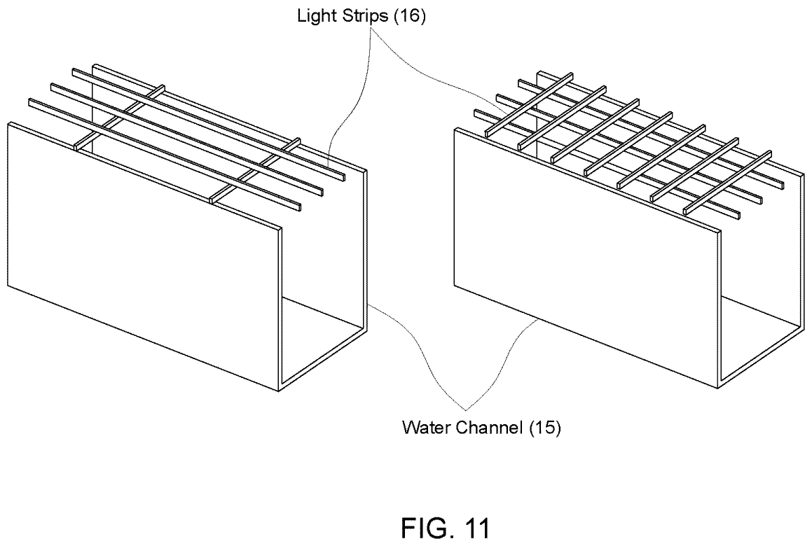

FIG. 11 Illustrates exemplary use of light strips 15 with open space in between above a water channel, or a tank or a vessel containing photo-aggregates to provide both man-made light and/or sun light when available, The sources of man-made light could be generated by incandescence, i.e., the emission of light from a heated object; electric discharge, i.e., light generated through the passage of an electric current through a gas or vapor; phosphorescence and fluorescence, i.e., light emitted when certain materials absorb electromagnetic radiation (such as UV light) and re-emit it at a longer wavelength; bioluminescence, i.e., production and emission of light by living organisms; chemiluminescence, i.e., light produced by a chemical reaction: light-emitting diodes (LEDs), i.e., light emitted when an electric current passing through a semiconductor device; and laser light generated through stimulated emission of radiation. These sources can typically generate wavelengths of lights in visible range of 380 nm to 750 nm, UVA range of 315 nm to 400 nm, UVB range of 280 to 315 nm, UVC range of 200 to 280 nm, and infrared light of 700 to 15,000 nm. These sources are energized by power suppliers, such as ballasts or drivers. Automatic controls are used to turn lights on and off, and to adjust light intensity depending on the light level required (e.g., intensity, duration of on/off cycle) in the process. For example, dimming lights or turning off segments of light strips 15 or entire light strips 15 when natural sun light is adequate to conserve energy. Mechanical wiping of light source 1 surfaces or spraying of a cleaning agent can be utilized to remove any fouling on these optical surfaces.

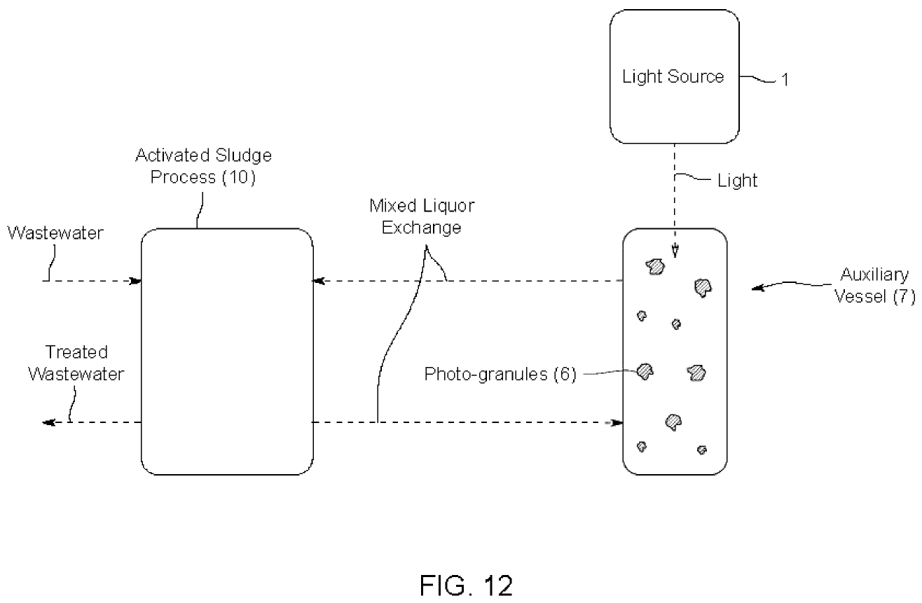

FIG. 12 is similar to FIG. 4A and further illustrates another embodiment of the present invention where an external light source 1 is used in the auxiliary vessel 7 to induce photo-granulation 6. As part of the mixed liquor exchange 8, the said tank may either be directly or not directly part of the activated sludge or biological culturing process 10. In one embodiment, the liquid is separated from a bulk-solids medium (using a liquid-solid separator) to improve clarity and to promote photo radiation, and this liquid, largely separated from the bulk is exposed to photo reactions (thus for external photoselection) to grow the requisite phototrophs before sending this liquid mass back to bioprocess in any preferred zone for the depolluting reaction. The liquid-solid separator, can be a classifier, a lamella, a clarifier, a filter, a membrane, as various example embodiments. Another approach is to use variable or intermittent mixers to allow for example the settlement of bulk solids (and thus effecting separation), exposure of liquid to light, and then the resumption of mixing in such manner of variable control or intermittent action. In one approach, mixing is used to bring the phototrophs in proximity to the light source 1, and jetting action or a directed flow may help simultaneously bring the phototrophs to the light source 1 while maintaining a clean surface. So rather than have quiescent conditions, a mixed approach may be facilitated as needed. Thus, mixing or lack thereof can be used to promote biofilms or photo-granules.

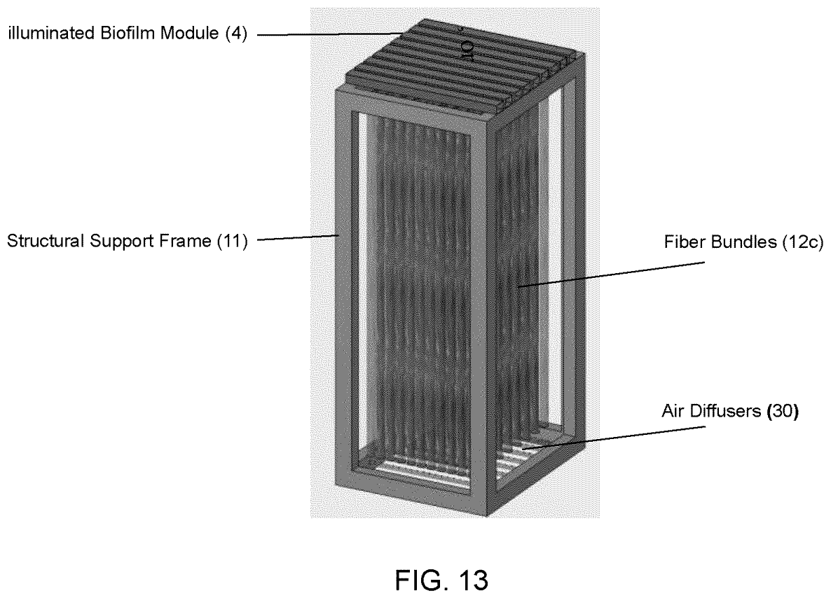

FIG. 13 illustrates an exemplary embodiment of the present invention including individual components previously described in FIG. 5 to FIG. 10, all integrated in one unit namely the light source and coupling including, FIG. 5 and FIG. 6, heat dissipation and controls FIG. 8 and FIG. 9, a supporting frame, FIG. 7, a set of fiber bundles FIG. 5 and FIG. 10, and means for mechanically controlling the biofilm here represented by air diffusers 30 as other mechanical means can be incorporated.

The terms granule, photo-granule, aggregate or densified solids may have different meanings but can be used interchangeably.

The foregoing descriptions of specific embodiments of the present disclosure have been presented for purposes of illustration and description. They are not intended to be exhaustive or to limit the present disclosure to the precise forms disclosed, and many modifications and variations are possible in light of the above teaching. The embodiments were chosen and described to best explain the principles of the present disclosure and its practical application, and to thereby enable others skilled in the art to best utilize the present disclosure and various embodiments with various modifications as are suited to the particular use contemplated. It is understood that various omissions and substitutions of equivalents are contemplated as circumstances may suggest or render expedient, but such omissions and substitutions are intended to cover the application or implementation without departing from the scope of the present disclosure.

What is claimed is:

1. An illuminated biofilm or photo-granule system 100 for enhancing wastewater treatment processes or biological culturing, the system 100 comprising:
a. a light source 1 having a wavelength that induce at least one phototrophic microorganism growth;
b. an optical coupling subsystem 2, the optical coupling subsystem 2 is coupled with the light source 1;
c. a light transmitting and scattering substratum 3;
d. one of a plurality of biofilm 4 or photo-granules 6 containing at least one phototrophic microorganism growing on the surface of the light transmitting and scattering substratum 3,
wherein the light source 1 and the optical coupling subsystem 2 are enclosed and connected directly to the light transmitting and scattering substratum 3 using electric controls, frames, supports, electric cables, optical cables, connectors, optical splitters, and other appurtenances.

2. The system of claim 1, further comprising an optional optical input connection 5 connected with one of: the optical coupling subsystem 2 and the light transmitting and scattering substratum 3.

3. The system of claim 2 wherein:
a. the optional optical input connection 5 conveys the light to the light transmitting and scattering substratum 3;
b. the light transmitting and scattering substratum 3 system induces light by illumination through the surface of the substratum forming an illuminated substratum 3a; and
c. the scattered light through the surface of the illuminated substratum 3a induces the growth of at least one phototrophic microorganism on the plurality of biofilm 4 or the photo-granules 6 growing on the illuminated substratum 3a.

4. The system of claim 3, wherein the illuminated substratum 3a is submerged in a biological wastewater treatment process tank, including an activated sludge or biological culturing process 10 tank.

5. The system of claim 4, wherein the illuminated substratum 3a is submerged in an auxiliary vessel 7, the auxiliary vessel 7 is hydraulically connected to a biological wastewater treatment process tank, and an active mixed liquor is exchanged within the biological wastewater treatment process tank and the auxiliary vessel 7.

6. The system of claim 5, wherein the depth of the plurality of biofilm 4 or photo-granules 6 is hydraulically controlled by inducing water or gas turbulence on the surface of the plurality of biofilm 4 or photo-granules 6 to induce erosion of the plurality of biofilm 4 or photo-granules 6.

7. The system of claim 3, wherein the illuminated substratum 3a is in the shape of a fiber, or a plurality of fibers, or a flat sheet, or a plurality of flat sheets, woven or non-woven matt, and fabric, or a plurality of woven or non-woven matts and fabrics.

8. The system of claim 3, wherein the mass transfer to the surface of the plurality of biofilm 4 or photo-granules 6 is controlled by continuous liquid movement on the surface of the plurality of biofilm 4 or photo-granules 6.

9. The system of claim 3, wherein light is supplied continuously, intermittently, or in pulses or to the illuminated substratum 3a.

10. The system of claim 1, wherein the wavelength of the light source 1 is selected to induce the growth of algae, or the growth of phototrophic bacteria, or phototrophic archaea.

11. The system of claim 1, wherein the light source 1 is selected from dimmable, intermittent based on a set pattern, operating in a pulse mode, controlled using sensors, using machine learning, and artificial intelligence approaches.

12. The system of claim 3, wherein material used within mobile biofilms or media from integrated fixed film activated sludge, including sponges, silicone, polyurethane or other inorganic or organic material (including cellulosic or hemicellulosic material), wherein the one or more materials aid in cleaning the illuminated substratum 3a or where the biofilm is used for biological culturing.

13. The system of claim 12, wherein the illuminated substratum 3a is further cleaned passively or actively, including optically, biologically, chemically, or physically.

14. The system of claims 1, wherein light irradiates into the tank or a vessel and the irradiated light is used for one of to:
a. select for microorganisms and morphologies that aggregate and thus increase transmissivity of light; and
inhibit microorganisms using photo deselection, and to grow such photo inhibited microorganisms sheltered away from the photo source and internally contained within dense morphologies,

15. The system of claims 1, wherein light irradiates into the tank or a vessel and
wherein the light irradiation occurs from an external light source 1 located above the water including, light strips 15 or within the water using an illuminated substratum 3a, or
wherein the selection results in the accumulation of storage products as an internal carbon source, including polyhydroxyalkanoate, glycogen, carbohydrates or proteins, for denitirification or phosphorus removal, or
wherein the selection is used to grow only sufficient phototrophs for producing oxygen necessary for aerobic reactions including for nitrification or deammonification or biological phosphorus removal.

16. The system of claim 14, wherein an active turbulence or mixing is used to control the size of the aggregates or to bring the aggregate to an illuminated substratum and the active turbulence is created by one of a pump, a mixer, a cyclone, baffles.

17. The system of claim 14, wherein the light irradiated to an auxiliary vessel 7, the auxiliary vessel 7 is hydraulically connected to a biological wastewater treatment process tank, and an active mixed liquor exchange 8 occurs with the biological wastewater treatment process tank and the auxiliary vessel 7.

18. The system of claim 17, wherein the mixed liquor is separated from a bulk-solids medium (using a liquid-solid separator, including a classifier, a lamella, a clarifier, a filter, a membrane) to improve clarity and to promote photo radiation.

19. A method for enhancing biological wastewater treatment or biological culturing, the method comprising:
a. providing an illuminated light source for biofilm or photo-granule, using an external light source 1;
b. submerging a light transmitting and scattering substratum 3 in a vessel where biological wastewater treatment or biological culturing takes place;
c. illuminating the light transmitting and scattering substratum 3 with a light capable of inducing the growth of phototrophic microorganisms forming an illuminated substratum 3a;
d. growing at least one phototrophic microorganism in a plurality of biofilm 4 ttached to the surface of the illuminated substratum 3a or photo-granules 6

20. The method of claim 19, wherein the plurality of biofilm 4 or photo-granules 6 results in:
accumulation of storage products as an internal carbon source, including polyhydroxyalkanoate, glycogen, carbohydrates or proteins, for denitirification or phosphorus removal, or
inducing growth of only sufficient phototrophs for producing oxygen necessary for aerobic reactions, including for nitrification or deammonification or biological phosphorus removal, or
inducing growth of phototrophic organisms, that consume directly or indirectly inorganic carbon including carbonic acid, or carbon dioxide or bicarbonate, or for pH management.
